(19) United States
(12) Patent Application Publication
(10) Pub. No.: US 2013/0216619 A1
(43) Pub. Date: Aug. 22, 2013

(54) PHARMACEUTICAL COMPOSITION OF ATORVASTATIN AND EZETIMIBE

(71) Applicant: Ranbaxy Laboratories Limited, (US)

(72) Inventors: Ashish RAGHUVANSHI, New Delhi (IN); Ankur BHARGAVA, Gurgaon (IN); Sachin ARORA, Gurgaon (IN); Ajay Kumar SINGLA, Gurgaon (IN)

(73) Assignee: RANBAXY LABORATORIES LIMITED, New Delhi (IN)

(21) Appl. No.: 13/766,051

(22) Filed: Feb. 13, 2013

(30) Foreign Application Priority Data
Feb. 16, 2012 (IN) 450/DEL/2012

(51) Int. Cl. A61K 9/24

(52) U.S. Cl. CPC A61K 9/209
USPC 424/463; 424/400; 424/475; 514/210.02; 424/490; 264/134; 427/2.14

(57) ABSTRACT

The present invention relates to an oral pharmaceutical composition comprising: a) a core comprising atorvastatin or a pharmaceutically acceptable salt thereof and an alkalizing agent; b) an intermediate coating over the core; and c) an outer coating comprising ezetimibe.

### FIELD OF THE INVENTION

The present invention relates to an oral pharmaceutical composition comprising: a) a core comprising atorvastatin or a pharmaceutically acceptable salt thereof and an alkalizing agent; b) an intermediate coating over the core; and c) an outer coating comprising ezetimibe.

### BACKGROUND OF THE INVENTION

Atorvastatin is susceptible to heat, moisture, low pH environment, and light. In an acidic environment, the hydroxy acid moiety present in atorvastatin converts to lactone. In addition, atorvastatin may be further destabilized in contact with the molecular moieties of other excipients during the formulation process. Since commonly used excipients such as binders, diluents, anti-adherents, and surfactants may adversely interact with atorvastatin, it is therefore necessary to add a stabilizer to the composition.

Various attempts have been made to stabilize atorvastatin. U.S. Pat. Nos. 5,686,104 and 6,126,971 disclose oral pharmaceutical formulations of atorvastatin in which the formulations are stabilized by the addition of a pharmaceutically acceptable alkaline earth metal salt.

Ezetimibe, chemically 1-(4-fluorophenyl)-3(R)-[3-(4-fluorophenyl)-3(S)-hydroxypropyl]-4(S)-(4-hydroxyphenyl)-2-azetidinone, is a cholesterol absorption inhibitor. The therapeutic uses of ezetimibe and related compounds, and their preparations are disclosed in U.S. Pat. No. 5,767,115. Ezetimibe is commercially available as 10 mg tablets. It is sold under the name Zetia®. Ezetimibe is available in the United States in a combination with simvastatin, sold under the trade name Vytorin®. However, ezetimibe is susceptible to alkaline hydrolysis as reported by Gajjar and Shah in The Open Conference Proceedings Journal, 2, p. 108-112 (2011).

There have been several reports in the literature on the combination of an HMG-CoA reductase inhibitor with ezetimibe. The HMG-CoA reductase inhibitor and ezetimibe combination is indicated as an adjunctive therapy to diet for the reduction of elevated total-C, LDL-C, Apo B, TG, and non-HDL-C levels, and to increase HDL-C levels in patients with primary (heterozygous familial and non-familial) hypercholesterolemia, or mixed hyperlipidemia. It is also indicated for the reduction of elevated total-C and LDL-C levels in patients with homozygous familial hypercholesterolemia as an adjunct to other lipid-lowering treatments.

U.S. Patent Publication No. 2002/0169134 discloses a pharmaceutical composition for the treatment or prevention of sitosterolemia comprising ezetimibe and lipid lowering agents, including atorvastatin.

U.S. Pat. No. 7,229,982 discloses a pharmaceutical composition comprising ezetimibe, simvastatin, BHA, and citric acid, wherein the composition is free of ascorbic acid.

PCT Publication No. WO 2006/134604 discloses a pharmaceutical composition of ezetimibe with various statins such as atorvastatin, simvastatin, and rosuvastatin. It discloses a tablet of ezetimibe and statin wherein all the excipients are blended with active ingredients, granulated, and compressed into suitable size tablets

PCT Publication No. WO 2009/024889 discloses a pharmaceutical composition comprising a first component comprised of granules of HMG-CoA reductase inhibitor and alkaline earth metal salt additive, and a second component comprising granules of ezetimibe.

The alkalizing agent used to stabilize atorvastatin formulation may degrade ezetimibe if both of them come in direct contact; therefore, there is a need in the prior art to develop a stable formulation comprising atorvastatin and ezetimibe.

The present invention discloses an alternate pharmaceutical composition comprising atorvastatin, an alkalizing agent, and ezetimibe, wherein ezetimibe and the alkalizing agent do not come in direct contact with each other.

### SUMMARY OF THE INVENTION

According to one aspect of the present invention, there is provided an oral pharmaceutical composition comprising:
a) a core comprising atorvastatin or a pharmaceutically acceptable salt thereof and an alkalizing agent;
b) an intermediate coating over the core; and
c) an outer coating comprising ezetimibe.

According to another aspect of the present invention, there is provided an oral pharmaceutical composition comprising:
a) a core comprising atorvastatin or a pharmaceutically acceptable salt thereof and an alkalizing agent selected from the group consisting of alkali metal salt additives, alkaline earth metal salt additives, an organic amine, or mixtures thereof;
b) an intermediate coating over the core; and
c) an outer coating comprising ezetimibe.

According to another aspect of the present invention, there is provided an oral pharmaceutical composition comprising:
a) a core comprising atorvastatin or a pharmaceutically acceptable salt thereof and an alkalizing agent selected from the group consisting of sodium carbonate, sodium bicarbonate, sodium hydroxide, sodium silicate, disodium hydrogen orthophosphate, sodium aluminate, calcium carbonate, calcium hydroxide, magnesium carbonate, magnesium hydroxide, magnesium silicate, magnesium aluminate, aluminum magnesium hydroxide, tromethamine, meglumine, or mixtures thereof;
b) an intermediate coating over the core; and
c) an outer coating comprising ezetimibe.

According to another aspect of the present invention, there is provided a tablet comprising:
a) a core comprising atorvastatin or a pharmaceutically acceptable salt thereof and an alkalizing agent;
b) an intermediate coating over the core; and
c) an outer coating comprising ezetimibe.

According to another aspect of the present invention, there is provided a process for the preparation of tablets comprising the steps of:
a) blending atorvastatin with an alkalizing agent and one or more pharmaceutically acceptable excipients;
b) optionally, granulating the blend of step a);
c) lubricating the blend of step a) or the granules of step b);
d) compressing the blend of step c) into a suitable sized tablet;
e) coating the tablet of step d) with a dispersion of pharmaceutically acceptable excipients to form an intermediate coating; and
f) coating the tablet of step e) with a dispersion or solution of ezetimibe, and other pharmaceutically acceptable excipients in a suitable solvent.

According to another aspect of the present invention, there is provided a process for the preparation of capsules comprising the steps of:
a) dispersing atorvastatin, an alkalizing agent, and one or more pharmaceutically acceptable excipients in a suitable solvent;
b) coating the dispersion of step a) onto non-pareils sugar beads;
c) coating the coated pellets of step b) with a dispersion of pharmaceutically acceptable excipients to form an intermediate coating;
d) coating the coated pellets of step c) with a dispersion or a solution of ezetimibe and other pharmaceutically acceptable excipients in a suitable solvent; and
e) filling the pellets of step d) in a suitable size capsule.

### DETAILED DESCRIPTION OF THE INVENTION

Atorvastatin, as used herein, may be present in the form of atorvastatin or pharmaceutically acceptable salts thereof, for example, calcium, magnesium, or potassium. Atorvastatin may exist in any of the solid state forms available such as amorphous, or any other polymorphic form, in particular, crystalline Form I.

As used herein, the term “ezetimibe” refers to any polymorphic form available of ezetimibe such as crystalline anhydrous, crystalline hydrous, or amorphous forms.

The pharmaceutical composition according to the present invention comprises a core comprising atorvastatin and an outer coating comprising ezetimibe. The outer coating is coated over the core that has been covered with an intermediate coating. The intermediate coating may cover the core fully or partially. The pharmaceutical composition may be in the form of a tablet or a capsule.

The core according to the present invention may comprise atorvastatin and an alkalizing agent. The said core may be in the form of tablets, granules, fine granules, or pellets. Atorvastatin may be coated onto an inert carrier to obtain the core. The inert carrier used may include readily available inert cores, for example, non-pareils sugar beads or microcrystalline cellulose beads.

The alkalizing agents, as used herein, may include alkali metal salt additives, alkaline earth metal salt additives, and organic amines. Alkali metal salt additives may be, for example, sodium carbonate, sodium bicarbonate, sodium hydroxide, sodium silicate, disodium hydrogen orthophosphate, sodium aluminate, or mixtures thereof. Akaline earth metal salt additives may include, for example, calcium carbonate, calcium hydroxide, magnesium carbonate, magnesium hydroxide, magnesium silicate, magnesium aluminate, aluminum magnesium hydroxide, or mixtures thereof. Organic amines may be, for example, tromethamine, meglumine, or mixtures thereof. The alkalizing agent may be present in an amount of about 20% to about 50% based on the core weight.

The core may further comprise other pharmaceutically acceptable excipients, for example, disintegrants, binders, surfactants, diluents, anti-oxidants, lubricants, glidants, or mixtures thereof.

Specific examples of disintegrants include crospovidone, low-substituted hydroxypropyl cellulose, croscarmellose sodium, sodium starch glycolate, starch, carmellose calcium, or mixtures thereof.

Examples of antioxidants include butylated hydroxy anisole, butylated hydroxy toluene, DL-alpha-tocopherol, ascorbic acid, sodium ascorbate, fumaric acid, or mixtures thereof.

Specific examples of binders include methyl cellulose, hydroxypropyl cellulose (HPC-L), methylcellulose, carboxymethyl cellulose sodium, hydroxypropyl methylcellulose, polyvinylpyrrolidone, or mixtures thereof.

Specific examples of diluents include lactose; mannitol; microcrystalline cellulose; cellulose-powdered; starch, for example, pregelatinized starch or maize starch; or mixtures thereof.

Specific examples of lubricants/glidants include colloidal silicon dioxide, stearic acid, magnesium stearate, calcium stearate, talc, hydrogenated castor oil, sucrose esters of fatty acid, microcrystalline wax, yellow beeswax, white beeswax, sodium stearyl fumarate, or mixtures thereof.

Specific examples of surfactants include polysorbate, sodium lauryl sulphate, polyethylene glycol, or mixtures thereof.

The tablets may be prepared by a direct compression method or by a granulation process. The granules can be prepared by dry granulation or wet granulation. The wet granulation may be carried out using granulating fluid or binder solution. The binder solution may comprise a suitable hydrophilic polymer dispersed or dissolved in a solvent. The dry granulation may be carried out by roller compaction or slugging.

The solvent used for granulation includes water, ethyl alcohol, isopropyl alcohol, acetone, or mixtures thereof.

The pharmaceutical composition further comprises an intermediate coating and an outer coating. The outer coating comprises ezetimibe and other pharmaceutically acceptable excipients. The intermediate coating is present between the core and the outer coating. The intermediate coating is deployed in order to prevent interactions between the alkalizing agent present in the core, and ezetimibe present in the outer coating. The intermediate coating may be present in an amount of about 1% to about 5% based on the core weight. The outer coating may be present in an amount of about 5% to about 15% based on the core weight.

The intermediate coating or the outer coating may comprise film-forming polymers and other pharmaceutically acceptable excipients.

Examples of film-forming polymers include ethyl cellulose, hydroxypropyl methylcellulose, methylcellulose, carboxy methylcellulose, hydroxymethyl cellulose; polyethylene glycol, methacrylic acid polymers such as Eudragit® RL and RS, xanthan gum, polyvinyl alcohol, or mixtures thereof. Alternatively, commercially available coating compositions comprising film-forming polymers marketed under various trade names such as Opadry® may also be used for coating.

The other pharmaceutically acceptable excipients present in the intermediate coating and the outer coating include plasticizers, for example, propylene glycol, triethyl citrate, tributyl citrate, dibutyl sebacate, acetyl tri butyl citrate, glyceryl monostearate, triacetin, polyethylene glycol, diethyl phthalate, acetylated monoglycerides, cetyl alcohol, or mixtures thereof; opacifiers, for example, titanium dioxide, silicon dioxide, talc, and behenic acid; antifoaming agents, for example, simethicone emulsion, dimethicone, and lutrol; and solvents, for example, water, ethanol, methanol, isopropyl alcohol, dichloromethane, acetone, or mixtures thereof.

The outer coating may further comprise surfactants, for example, sodium lauryl sulfate, polysorbates, and polyethylene glycols.

Additionally, the outer coating may be further coated with a non-functional coating.

The coating may be performed by using any conventional coating technique known in the art such as spray coating in a conventional coating pan or fluidized bed processor, or dip coating.

According to one of the embodiments, there is provided a process for the preparation of tablets comprising the steps of:
a) blending atorvastatin with an alkalizing agent and one or more pharmaceutically acceptable excipients;
b) granulating the blend of step a);
c) lubricating the granules of step b);
d) compressing the blend of step c) into a suitable size tablet;
e) coating the tablet of step d) with a dispersion of pharmaceutically acceptable excipients to form an intermediate coating; and
f) coating the tablet of step e) with a dispersion or solution of ezetimibe, a surfactant, and other pharmaceutically acceptable excipients in a suitable solvent.

According to another embodiment, there is provided a process for the preparation of capsules comprising the steps of:
a) dispersing atorvastatin, an alkalizing agent, and one or more pharmaceutically acceptable excipients in a suitable solvent;
b) coating the dispersion of step a) onto non-pareils sugar beads;
c) coating the coated pellets of step b) with a dispersion of pharmaceutically acceptable excipients to form an intermediate coating;
d) coating the coated pellets of step c) with a dispersion or solution of ezetimibe, a surfactant, and other pharmaceutically acceptable excipients in a suitable solvent; and
e) filling the pellets of step d) into a suitable size capsule.

The following example illustrates the invention but does not limit the scope of the invention.

### EXAMPLE 1

| Excipients | Quantity (mg/tablet) |
|---|---|
| Core (Intragranular) | |
| Atorvastatin calcium eq. to atorvastatin | 86.77 |
| Pregelatinized starch | 120.00 |
| Microcrystalline cellulose | 121.43 |
| Lactose monohydrate | 127.00 |
| Calcium carbonate | 265.20 |
| Croscarmellose sodium | 24.00 |
| Granulation Solution | |
| Hydroxypropyl cellulose | 16.00 |
| Polysorbate 80 | 6.40 |
| Purified water | q.s. |
| Extragranular | |
| Croscarmellose sodium | 24.00 |
| Colloidal silicon dioxide | 5.20 |
| Magnesium stearate | 4.00 |
| Intermediate Coating | |
| Opadry ® | 24.00 |
| Purified water | q.s. |
| Outer Coating | |
| Ezetimibe | 10.00 |
| Hydroxypropyl methylcellulose | 20.00 |
| Talc | 2.00 |
| Sodium lauryl sulfate | 0.08 |
| 30% Simethicone emulsion | 0.39 |
| Purified water | q.s. |
| Film Coating | |
| Opadry ® | 17.13 |
| Purified water | q.s. |

### Manufacturing Process:

a) Atorvastatin, pregelatinized starch, microcrystalline cellulose, lactose monohydrate, calcium carbonate, and a part of croscarmellose were blended together.
b) Hydroxypropyl cellulose and polysorbate 80 were dispersed in water.
c) The blend of step a) was granulated with the dispersion of step b).
d) The granules of step c) were blended with the remaining part of croscarmellose.
e) The blend of step d) was lubricated with colloidal silicon dioxide and magnesium stearate.
f) The blend of step e) was compressed into a suitable size tablet.
g) The tablet of step f) was coated with a dispersion of Opadry® in water.
h) Ezetimibe, hydroxypropyl methylcellulose, talc, sodium lauryl sulfate, and simethicone emulsion were dispersed in water.
i) The coated tablet of step g) was coated with the dispersion of step h).
j) The coated tablet of step i) was coated with the dispersion of Opadry® in water.

We claim:

1. An oral pharmaceutical composition comprising:
a) a core comprising atorvastatin or a pharmaceutically acceptable salt thereof and an alkalizing agent;
b) an intermediate coating over the core; and
c) an outer coating comprising ezetimibe.

2. The oral pharmaceutical composition of claim 1, wherein the alkalizing agent is selected from the group consisting of alkali metal salt additives, alkaline earth metal salt additives, an organic amine, or mixtures thereof.

3. The oral pharmaceutical composition according to claim 1, wherein the alkalizing agent is selected from the group consisting of sodium carbonate, sodium bicarbonate, sodium hydroxide, sodium silicate, disodium hydrogen orthophosphate, sodium aluminate, calcium carbonate, calcium hydroxide, magnesium carbonate, magnesium hydroxide, magnesium silicate, magnesium aluminate, aluminum magnesium hydroxide, tromethamine, meglumine, or mixtures thereof.

4. The oral pharmaceutical composition according to claim 2 or 3, wherein the alkalizing agent is present in an amount of about 20% to about 50% based on the core weight.

5. The oral pharmaceutical composition according to claim 1, wherein the core may be in the form of tablets, granules, fine granules, or pellets.

6. The oral pharmaceutical composition according to claim 1, wherein the core further comprises other pharmaceutically acceptable excipients selected from the group consisting of disintegrants, binders, surfactants, diluents, anti-oxidants, lubricants, glidants, or mixtures thereof.

7. The oral pharmaceutical composition according to claim 1, wherein the intermediate or outer coating comprises a film-forming polymer and other pharmaceutically acceptable excipients.

8. The oral pharmaceutical composition according to claim 1, wherein the pharmaceutical composition may be in the form of capsules.

9. The oral pharmaceutical composition according to claim 8, prepared by the process comprising the steps of:
a) dispersing atorvastatin, an alkalizing agent, and one or more pharmaceutically acceptable excipients in a suitable solvent;
b) coating the dispersion of step a) onto non-pareils sugar beads;
c) coating the coated pellets of step b) with a dispersion of pharmaceutically acceptable excipients to form an intermediate coating;
d) coating the coated pellets of step c) with a dispersion or solution of ezetimibe and other pharmaceutically acceptable excipients in a suitable solvent; and
e) filling the pellets of step d) into a suitable size capsule.

10. The oral pharmaceutical composition according to claim 1, wherein the pharmaceutical composition may be in the form of tablets.

11. The oral pharmaceutical composition according to claim 10, prepared by the process comprising the steps of:
a) blending atorvastatin with an alkalizing agent and one or more pharmaceutically acceptable excipients;
b) optionally, granulating the blend of step a);
c) lubricating the blend of step a) or the granules of step b);
d) compressing the blend of step c) into a suitable size tablet;
e) coating the tablet of step d) with a dispersion of pharmaceutically acceptable excipients to form an intermediate coating; and
f) coating the tablet of step e) with a dispersion or solution of ezetimibe in a suitable solvent along with other pharmaceutically acceptable excipients.
